(19) United States
(12) Patent Application Publication
(10) Pub. No.: US 2025/0360251 A1
(43) Pub. Date: Nov. 27, 2025

(54) METHOD AND DEVICE FOR MEASURING OXYGEN SATURATION

(71) Applicants: THE CHILDREN'S HOSPITAL OF PHILADELPHIA, Philadelphia, PA (US); VITARA BIOMEDICAL, INC., Philadelphia, PA (US)

(72) Inventors: Marcus Graeme DAVEY, Ardmore, PA (US); Matthew SLIPENCHUK, Chalfont, PA (US); Bartosz JASKULSKI, Turnersville, PA (US); Malena FARBER, Philadelphia, PA (US); Julia KNIPE, Olney, MD (US)

(21) Appl. No.: 18/867,163

(22) PCT Filed: May 22, 2023
(86) PCT No.: PCT/US2023/067310

§ 371 Date: Nov. 19, 2024

Related U.S. Application Data
(60) Provisional application No. 63/365,039, filed on May 20, 2022.

(51) Int. Cl. A61M 1/14; G01N 21/31; G01N 33/49

(52) U.S. Cl. CPC A61M 1/14; G01N 21/31; G01N 33/4925; A61M 2205/3313; A61M 2230/205; A61M 2240/00

(57) ABSTRACT

A system configured to enclose a premature fetus within an extracorporeal environment to promote growth of the fetus and increase viability of the fetus. The system includes a device for measuring oxygen saturation.

### CROSS-REFERENCE TO RELATED APPLICATIONS

This application claims the benefit of U.S. Provisional Application Ser. No. 63/365,039 filed May 20, 2022, the contents of which is hereby incorporated by reference as if set forth in its entirety herein.

### TECHNICAL FIELD

The present disclosure relates generally to neonatal care. More specifically, the present disclosure describes devices, systems, and methods related to improving the viability of a premature fetus outside of the womb. According to one aspect, the present disclosure relates to improving viability of premature fetuses at a stage of development prior to 28 weeks gestation and measuring oxygen saturation of the premature fetus. In another aspect, there is provided a system and method for measuring oxygen saturation in the blood of an animal non-invasively.

### BACKGROUND

Extreme prematurity is the leading cause of infant morbidity and mortality in the United States, with over one third of all infant deaths and one half of cerebral palsy diagnoses attributed to prematurity. The 2010 Center for Disease Control National Vital Statistics Report notes birth rates at a gestational age of less than 28 weeks in the United States over roughly the past decade have remained stable at approximately 0.7%, or 30,000 births annually. Similarly, birth rates at gestational ages 28-32 weeks over the past decade in the United States have been stable at 1.2%, or 50,000 births annually.

Premature birth may occur due to any one of a multitude of reasons. For example, premature birth may occur spontaneously due to preterm rupture of the membranes (PROM), structural uterine features such as shortened cervix, secondary to traumatic or infectious stimuli, or due to multiple gestation. Preterm labor and delivery is also frequently encountered in the context of fetoscopy or fetal surgery, where instrumentation of the uterus often stimulates uncontrolled labor despite maximal tocolytic therapy.

Respiratory failure represents the most common and challenging problem associated with extreme prematurity, as gas exchange in critically preterm neonates is impaired by structural and functional immaturity of the lungs. Advances in neonatal intensive care have achieved improved survival and pushed the limits of viability of preterm neonates to 22 to 24 weeks gestation, which marks the transition from the canalicular to the saccular phase of lung development. Although survival has become possible, there is still a high rate of chronic lung disease and other complications of organ immaturity, particularly in fetuses born prior to 28 weeks gestation. The development of a system that could support normal fetal growth and organ maturation for even a few weeks could significantly reduce the morbidity and mortality of extreme prematurity and improve quality of life in survivors.

The development of an “artificial placenta” has been the subject of investigation for over 50 years with little success. Previous attempts to achieve adequate oxygen saturation of the fetus in animal models have employed traditional extracorporeal membrane oxygen saturation (ECMO) with pump support and have been limited by circulatory overload and cardiac failure in treated animals. The known systems have suffered from unacceptable complications, including: 1) progressive circulatory failure due to after-load or pre-load imbalance imposed on the fetal heart by oxygenator resistance or by circuits incorporating various pumps; and 2) contamination and fetal sepsis.

There are a variety of methods and devices for measuring oxygen saturation levels in the blood presently on the market. Red blood cells contain hemoglobin molecules through which oxygen binds to the heme on the hemoglobin molecule. These devices typically measure the level of oxygen of arterial, oxygenated blood in the body. It is important to note that fetal hemoglobin levels differ from those of adult. This is due to the differences in the subunits of hemoglobin between fetus and adults; fetal hemoglobin has a higher affinity for oxygen and will not release oxygen to the tissues as readily.

Accordingly, a system and method configured to provide extracorporeal support for a premature fetus, or fetuses (preterm or term) with adequate respiratory gas exchange to support life, due to a spectrum of conditions/disorders, may improve viability. A system and method for measuring oxygen saturation may also improve viability.

### SUMMARY

A sensor system for measuring oxygen saturation in blood, such as a premature fetus's blood in an ex-utero environment, can include a light source configured to emit a light wave, a light sensor configured to sense a light wave; and a control unit. The control unit can include at least one memory having instructions stored therein that, upon execution by the control unit, cause the sensor system to perform operations comprising: emitting at least one light wave from the light source, receiving a reflected light wave with the light sensor, and comparing a parameter of the reflected light wave to a parameter of the at least one light wave to determine the oxygen saturation in the blood of the fetus.

The sensor system can include an oxygenator in fluid communication with the fetus so as to receive blood from the fetus. The sensor system can determine the oxygen saturation of the blood of the fetus within the oxygenator. The oxygenator can introduce oxygen into the blood of the fetus. The control unit can modify an amount of oxygen supplied to the oxygenator to modify an amount of oxygen introduced into the blood of the fetus by the oxygenator. The light source can emit a first light wave at a first wavelength and emit a second light wave at a second wavelength different from the first wavelength. The at least one light wave can have a wavelength of about 400 nanometers to about 700 nanometers. The light sensor can sense a first reflected light wavelength and a second reflected light wavelength different from the first wavelength. The light sensor can receive the first and second reflected light wavelengths in response to the light source emitting a single light wave at a selected wavelength.

The at least one memory can have instructions stored therein that, upon execution by the control unit, causes the sensor system to perform operations comprising comparing a parameter of the reflected light wave to one or more stored values to determine the oxygen saturation in the blood of the fetus. The sensor system can sense oxygen saturation levels that range from about 30% to about 100%.

A method for measuring oxygen saturation in a blood of the fetus in an ex-utero environment can include connecting a premature fetus to an ex-utero system configured to provide oxygen to the fetus, wherein the connecting step comprises the steps of attaching a first cannula to a vein of an umbilical cord, attaching a second cannula to an artery of the umbilical cord and connecting one or more of the first and second cannulae to an oxygenator such that blood is delivered from the fetus to the oxygenator and blood is delivered from the oxygenator to the fetus. The method can include emitting, by a light source, a light wave toward the blood of the fetus, sensing, by a light sensor, a reflected light wave reflected by the blood of the fetus, and comparing, by a control unit, a parameter of the reflected light wave to one or more stored values to determine the oxygen saturation in the blood of the fetus.

Emitting the light wave can include emitting a light wave at a wavelength of about 400 nanometers to about 700 nanometers. The method can include determining the oxygen saturation in the blood of the fetus within the oxygenator. The method can include determining the oxygen saturation in the blood of the fetus without infrared light. The light source and the light sensor can each be positioned on a same side of the oxygenator. The method can include modifying, by the control unit, an amount of oxygen supplied to the oxygenator thereby modifying the amount of oxygen introduced into the blood of the fetus by the oxygenator. The sensing step can include sensing, by the light sensor, a first reflected light wavelength and a second reflected light wavelength different from the first wavelength. The reflected light wave sensed in the sensing step can be a first reflected light wave at a first wavelength and the method can include sensing, by the light sensor, a second reflected light wave at a second wavelength. The first and second light waves can be a reflection of the light wave emitted by the light source.

### BRIEF DESCRIPTION OF THE DRAWINGS

The foregoing summary, as well as the following detailed description of illustrative embodiments of the application, will be better understood when read in conjunction with the appended drawings. For the purposes of illustrating the present disclosure, there is shown in the drawings illustrative embodiments. It should be understood, however, that the application is not limited to the specific embodiments and methods disclosed, and reference is made to the claims for that purpose. In the drawings:

FIG. 1 illustrates one embodiment of a device and system for measuring oxygen saturation;

FIG. 2 illustrates a perspective view of an oxygenator for use with the system of FIG. 1; and

FIG. 3 illustrates a schematic drawing of a controller and the device of FIG. 1.

FIG. 4 compares the actual oxygen saturation (SaO₂) to predicted oxygen saturation using the device and method of FIG. 1.

### DETAILED DESCRIPTION OF ILLUSTRATIVE EMBODIMENTS

Aspects of the disclosure will now be described in detail with reference to the drawings, wherein like reference numbers refer to like elements throughout, unless specified otherwise. Certain terminology is used in the following description for convenience only and is not limiting. The term “plurality”, as used herein, means more than one. The terms “a portion” and “at least a portion” of a structure include the entirety of the structure. Certain features of the disclosure which are described herein in the context of separate embodiments may also be provided in combination in a single embodiment. Conversely, various features of the disclosure that are described in the context of a single embodiment may also be provided separately or in any sub-combination.

Described herein is a sensor system that can detect oxygen saturation levels within a fluid. The sensor system can detect oxygen saturation levels in a fluid. The fluid can be the blood of an animal. The fluid can be mammalian blood. The fluid can be human blood. The fluid can be the blood of a premature human fetus. The sensor system can detect oxygen saturation levels without contacting the fluid. The sensor system can utilize visible wavelengths of light to determine the oxygen saturation of a blood sample. The sensor system can detect oxygen saturation levels without utilizing infrared light. The sensor system can detect oxygen saturation levels without contacting the blood sample. The sensor system can detect oxygen saturation levels of blood within an oxygenator. In one particular aspect, the sensor system can determine oxygen saturation in a premature fetus's blood in an ex-utero environment. The sensor system can detect oxygen in real time. The sensor system can continuously determine the oxygen saturation of a blood sample.

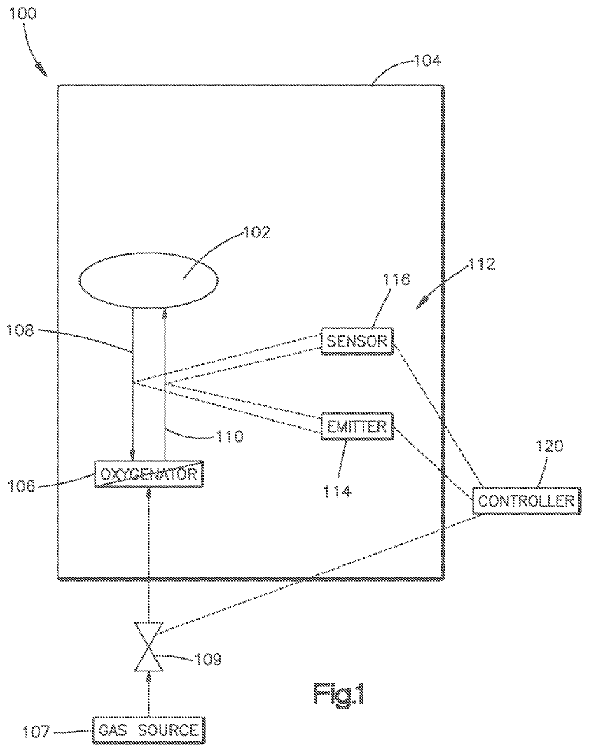

Referring to FIG. 1, a system 100 configured to provide extracorporeal support to a premature fetus 102 is shown. According to one aspect of the disclosure the system 100 is configured to provide a system environment that is similar to an environment the premature fetus 102 would experience in utero. Viability of a premature fetus that is removed from the uterine environment and that is, for example, between about 22 weeks to about 24 weeks gestation, may be increased by placing the premature fetus 102 in the system environment. Some non-limiting examples of extracorporeal systems suitable for the treatment of the premature fetus described herein are found in the following: US Publ. No. 2021/0052453 entitled “Extracorporeal Life Support System and Methods of Use Thereof”; US Publ. No. 2021/0161744 entitled “Method And Apparatus For Extracorporeal Support Of Premature Infants”; and US Publ. No. 2023/0000706 entitled “System and Method Configured to Provide Extracorporeal Support for Premature Fetus” which are incorporated herein by reference in their entirety.

The system 100 can include a housing 104 that defines an interior space to receive the fetus 102. A method of moving a premature fetus 102 from the uterus of a patient to the interior space of an ex-utero environment can include accessing and cannulating umbilical cord vessels (2 arteries and one vein) of the fetus 102, and connecting the cannulas to an oxygenator 106. The connecting step includes the step of attaching the fetus 102 to the oxygenator 106 such that deoxygenated blood is delivered from the fetus 102 to the oxygenator 106, and oxygenated blood is delivered from the oxygenator 106 to the fetus 102. An exemplary embodiment of oxygenator 106 is shown in pending application PCT US 2022/043259, which is incorporated herein by reference in its entirety. According to one aspect of the disclosure, the method may include, before the attaching step, the step of priming the oxygenator 106, for example with blood. One or more gasses (e.g., oxygen, carbon, nitrogen,) can be sent from a gas source 107 to the oxygenator 106 such that the oxygenator 106 introduces oxygen into the blood. A valve 109 can regulate the amount of gas supplied from the gas source 107 to the oxygenator 106. The valve 109 can be a ball valve, control valve, butterfly valve, or globe valve. In some examples, the valve 109 can be adjusted (e.g., moved toward an open position or closed position) in response to receiving an electrical signal. In such an embodiment, the valve is in electrical communication with a controller 120

The step of cannulating the fetus 102 may include the steps of: attaching a first cannula 108 to one of a vein and a first artery of the umbilical cord, and attaching a second cannula 110 to the other of the vein and the first artery of the umbilical cord. The step of cannulating the fetus can include attaching a third cannula to a second artery of the umbilical cord. The method may further include the step of connecting one or more of the first, second and third cannulae to an oxygenation circuit, which includes the oxygenator 106. The first cannula 108 can be in fluid communication with an inlet of the oxygenator 106. The second cannula 110 can be in fluid communication with an outlet of the oxygenator 106. One non-limiting example of a cannula contemplated for use is disclosed in U.S. patent application Publication Number 2021/0338270 titled “Cannula Insertion System And Methods Of Using The Same,” which is incorporated herein by reference in its entirety.

Each of the first and second cannulae 108, 110 can include a first end and a second end opposite the first end in a first direction. The first and second ends of the first cannula 108 can be fluidly coupled to the fetus 102 and an inlet of the oxygenator 106, respectively. The first and second ends of the second cannula can be fluidly coupled to the fetus 102 and an outlet of the oxygenator 106, respectively. Each of the first and second cannulae 108, 110 can include first and second sides opposite each other in a second direction perpendicular to the first direction.

A sensor system 112 can measure oxygen saturation in the blood of the fetus 102. The sensor system 112 can include an oximeter (also referred to herein as “pulse ox”) that utilizes visible wavelengths of light to determine the oxygen saturation of a blood sample. The sensor system 112 can determine oxygen saturation without directly contacting a blood sample, lowering the risk of infection and patient disruption. In some examples, the sensor system 112 comprises a transmissive oximeter. In other examples, the sensor system 112 comprises a reflective oximeter. In transmissive oximetry, a sensor (e.g., photodiode) and a light source are positioned on opposite sides of a measurement site. The light is emitted from the light source, transmitted through the measurement site, and received by the sensor. In reflective oximetry, both the sensor and the light source are on the same side of the measurement site. The light is emitted from the light source, reflected by the blood, and received by the sensor. Reflective oximetry can be utilized for measurement sites having increased depth and/or density compared to transmissive oximetry sites.

Referring to FIG. 1, the sensor system 112 can include an emitter 114 and a sensor 116. The emitter 114 can emit a wave (e.g., a light wave) toward at least one of the first and second cannulae 108, 110. The sensor 116 can detect a reflected portion of the wave. The emitter 114 and the sensor 116 can each be positioned on the first side of the first and second cannulae 108, 110. The emitter 114 and the sensor 116 can each be positioned on the same side of the oxygenator 106. The sensor system 112 can use reflective oximetry when the emitter 114 and the sensor 116 are each positioned on the same side of the oxygenator 106.

The emitter 114 can be a light source that emits light configured to be reflected and sensed by the sensor 116. The emitter 114 can be a light emitting diode (“LED”) array. The emitter 114 can include a plurality of light sources. The light emitter 114 can emit white light. The emitter 114 can emit light having a wavelength ranging from about 400 nanometers to about 1 millimeter, about 400 nanometers to about 800 nanometers, about 700 nanometers to about 1 millimeter, about 450 nanometers to about 600 nanometers, about 400 nanometers to about 500 nanometers, or about 600 nanometers to about 800 nanometers. The emitter 114 can emit white light. The emitter 114 can emit red light. The emitter 114 can emit green light. The emitter 114 can emit blue light. The emitter 114 can emit infrared light. The emitter 114 can emit a combination of light wavelengths. The emitter can emit a combination of red, green, and blue lights to provide a white light. The emitter 114 can emit a first light wave having a wavelength of about 625 nanometers to about 775 nanometers. The emitter 114 can emit a second light wave having a wavelength of about 475 nanometers to about 600 nanometers. The emitter 114 can emit a third light wave having a wavelength of about 400 nanometers to about 500 nanometers. The emitter 114 can include a plurality of LEDs. The emitter 114 can include an array of LEDs. In one particular embodiment, the emitter 114 can include four LEDs arranged in an array.

The sensor 116 can be configured to sense one or more light waves. The sensor 116 can be configured to sense reflected light waves. The sensor 116 can be configured to sense light waves emitted by the emitter 114 and reflected by blood of the fetus. The sensor 116 can be configured to sense a plurality of light waves at different wavelengths. The sensor 116 can be configured to simultaneously sense a plurality of light waves at different wavelengths. The sensor 116 can be configured to sense a first light having a wavelength of about 625 nanometers to about 775 nanometers. The sensor 116 can be configured to sense a second light wave having a wavelength of about 475 nanometers to about 600 nanometers. The sensor 116 can be configured to sense a third light wave having a wavelength of about 400 nanometers to about 500 nanometers. The sensor 116 can be configured to sense red light. The sensor 116 can be configured to sense blue light. The sensor 116 can be configured to sense green light. Some sensors contemplated for use in the sensor system 112 is the AS7341 Spectral Sensor from Adafruit Industries LLC and the TCS230 sensor.

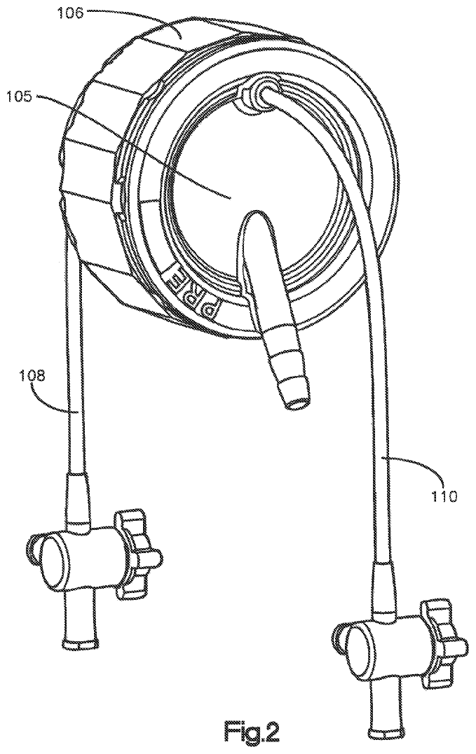

The sensor system 112 can be configured to determine the oxygen saturation of blood within the oxygenator 106. Referring to FIG. 2, the oxygenator 106 can include a transparent face 105 such that light can pass through the face 105. The emitter 114 can be positioned adjacent the face 105 and emit a light wave through the transparent face 105 such that light is reflected off the blood within the oxygenator 106. The sensor 116 can be positioned adjacent the face 105 so as to sense the light reflected from the blood within the oxygenator 106. In some examples, at least one of the sensor 116 and the emitter 114 are coupled to the oxygenator 106. In some examples, at least one of the sensor 116 and the emitter 114 are fixed to the oxygenator 106. The sensor 116 and the emitter 114 can be fixed to the transparent face 105 of the oxygenator 106.

The sensor system 112 can include a controller 120 configured to send and receive electrical signals. For example, the controller 120 can send and receive signals from at least one of the sensor 116 and the emitter 114. The controller 120 can send an emitter signal so as to cause the emitter 114 to emit the light wave. The emitter signal can include a parameter of the emitted light wave. The parameter can include the length of time the light wave is emitted. The parameter can include the intensity of the light wave emitted. The parameter can include the wavelength at which the light wave is emitted. The parameter can include the number of light waves emitted. The parameter can include the number of lumens emitted. The amount of light emitted can also be referred to as the number of lumens emitted.

The sensor 116 can send a sensor signal to the controller 120. In some examples, the sensor 116 sends the sensor signal directly to the controller 120. In other examples, the sensor 116 sends the sensor signal to one or more intermediate components that send a signal to the controller 120 in response to receiving the sensor signal. The sensor signal can include one or more parameters indicative of a property of the reflected light wave. The parameter can be indicative of the amount of reflected light sensed by the sensor 116. The parameter can be indicative of the number of lumens of reflected light sensed by the sensor 116. The parameter can be indicative of the number of wavelengths sensed by the sensor 116. The parameter can be indicative of the wavelength of each light wave sensed by the sensor 116. The parameter can be indicative of the length of time over which the light wave was sensed. The sensor signal can indicate the amount of each light wavelength sensed by the sensor 116.

The wavelength of the reflected light sensed by the sensor 116 can be indicative of the oxygen saturation. The sensor 116 can sense red, green, and blue wavelengths within the reflected light. The sensor signal can include the number of lumens for each of the red, green, and blue wavelengths. The number of lumens for the red, green, and blue wavelengths can be indicative of the oxygen saturation.

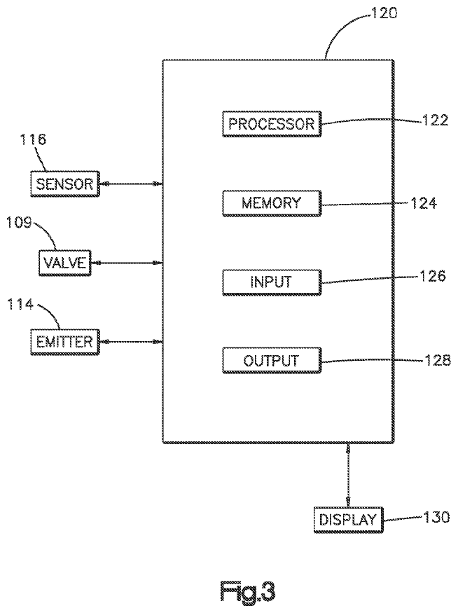

Referring to FIG. 3, the controller 120 can include a processor 122 and at least one memory 124. The at least one memory 124 can have instructions stored therein that cause the sensor system 112 to perform operations including emitting at least one light wave from the emitter 114, receiving a reflected light wave with the light sensor, and comparing a parameter of the reflected light wave to one or more stored values to determine the oxygen saturation of the blood of the fetus. The controller 120 can include an input 126 that receives the sensor signal from the sensor 116. The controller 120 can include an output 128 that sends an electrical signal. For example, the output 128 can send the emitter signal to the emitter 114. The controller 120 can store a parameter of the emitted light wave in the at least one memory. The controller 120 can store a parameter of the reflected light wave in the at least one memory.

The processor 122 can compare the parameters of the emitted and reflected light waves to each other to determine the amount of light absorbed by the blood of the fetus. The amount of light absorbed by the blood of the fetus 102 can be determined from the amount of light emitted by the emitter 114 compared to the amount of reflected light sensed by the sensor. The at least one memory 124 can include a table of values indicative of oxygen saturation in blood. The processor 122 can compare the amount of light absorbed by the blood of the fetus 102 to the table values so as to determine the oxygen saturation of the blood of the fetus 102. The processor 122 can compare the number of lumens of each of the red, green, and blue wavelengths sensed by the sensor116 to the table values. In some examples, the table values can be created by comparing different levels of oxygen saturation determined by the sensor system 112 to corresponding levels of oxygen saturation as determined by other existing systems. The processor 122 can compare the oxygen saturation of the blood to a threshold. The sensor system 112 can determine oxygen saturation levels ranging from about 30% to about 100%, about 30% to about 45%, about 45% to about 60%, about 60% to about 75%, about 75% to about 90%, or about 90% to about 100% in the blood of the fetus 102. The sensor system 112 can sense the oxygen saturation of the fetus's blood at the inlet and outlet of the oxygenator 106. The processor 122 can determine the amount of oxygen absorbed by the fetus 102 by comparing the oxygen saturation of the blood at the inlet and outlet of the oxygenator 106. The processor 122 can determine the oxygen saturation of the blood in real time. The processor 122 can continuously determine the oxygen saturation of the blood as the blood flows through the oxygenator.

The controller 120 can send a valve signal so as to adjust an amount of gas supplied from the gas source 107 to the oxygenator 106. In some examples, the controller 120 sends the valve signal directly to the valve 109. In other examples, the controller 120 sends the valve signal to one or more intermediate components that send a signal to the valve 109 in response to receiving the valve signal. The controller 120 can send the valve signal to increase the gas supplied to the oxygenator if the oxygen saturation is below the threshold. The controller 120 can send the valve signal to decrease the gas supplied to the oxygenator if the oxygen saturation is above the threshold.

The sensor system 112 can include a display 130 configured to display information regarding the oxygen saturation of the blood of the fetus 102. The controller 120 can send a display signal so as to cause the display 130 to display the information. In some examples, the controller 120 sends the display signal directly to the display 130. In other examples, the controller 120 sends the display signal to one or more intermediate components that send a signal to the display 130 in response to receiving the display signal. The display 130 can be a monitor, television, or other electronic display. The controller 120 can send the display signal to a computer. The controller 120 can store the oxygen saturation levels in the at least one memory. The display signal can cause the display 130 to display the sensed oxygen saturation levels in real time. In other examples, the display signal can cause the display 130 to display an average oxygen saturation level over a given time period. The time period can be about 1 second to about 10 seconds, about 1 second to about 30 seconds, about 30 seconds to about 1 minute, about 1 minute to about 5 minutes, or less than about 10 minutes.

Some oximeters emit a first light wave and a second light wave having a different wavelength than the first light wave. The first light wave can be a red light wave having a wavelength of approximately 660 nm. The second light wave can be an infrared light having a wavelength of approximately 940 nm. The light waves can be emitted sequentially such that only one of the first and second light waves are emitted at a time. However, this can require multiple light sources and the sequential emission can increase the time necessary to determine oxygen saturation levels. The sensor system 112 can emit a light from a single light source and the sensor 116 can sense multiple wavelengths simultaneously.

### EXAMPLES

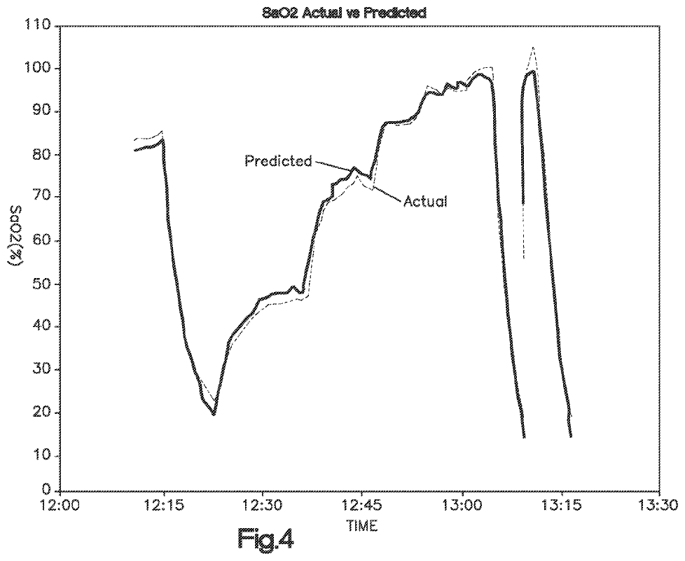

One experiment was run utilizing an experimental setup like the embodiment shown in FIG. 1 but without the fetus and housing. Instead, a continuous flow of animal blood was used in a closed loop system with a pump and a reservoir coupled to first and second cannulae coupled to an oxygenator. The blood was oxygenated with a Maquet M4 oxygenator. The blood within the closed loop system was tested to with a reference device and the sensor system 112 to determine the actual oxygen saturation vs. predicted oxygen saturation in a blood sample. Referring to FIG. 4, actual oxygen saturation was measured using a reference device. The reference device was a Spectrum Medical (“Through the tube”) from the manufacturer's website. The reference device determines O₂ saturation by analyzing a specific region of the oxy-hemoglobin absorption curve ranging from about 30% to about 100%. The reference device utilized a miniature scanning spectrometer and infrared LED to measure reflected amplitude of light at 100 discrete wavelengths to calculate an actual O2 concentration.

The current system uses a spectral sensor to “read color” of the blood through the oxygenator faceplate (although other areas of the blood circuit could be used) to generate RGB values. A linear model was developed to translate RGB into saturation values. The oxygen saturation levels predicted by the sensor system 112 were then compared to the actual oxygen saturation values from the reference device to confirm the accuracy of the sensor system 112.

It will be appreciated that the foregoing description provides examples of the disclosed system and methods. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

Recitation of ranges of values herein are merely intended to serve as a shorthand method of referring individually to each separate value falling within the range including the stated ends of the range, unless otherwise indicated herein, and each separate value is incorporated into the specification as if it were individually recited herein. All methods described herein can be performed in any suitable order unless otherwise indicated herein or otherwise clearly contradicted by context. The term “about” as used herein in reference to numerical ranges can mean the stated value +/−1%, 2%, 3%, 4%, 5% or 10%.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. As one of ordinary skill in the art will readily appreciate from the disclosure of the present invention, processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. A sensor system for measuring oxygen saturation in a blood sample, the sensor system comprising:
a light source configured to emit a light wave;
a light sensor configured to sense a light wave; and
a control unit, wherein the control unit includes at least one memory having instructions stored therein that, upon execution by the control unit, cause the sensor system to perform operations comprising:
emitting at least one light wave from the light source;
receiving a reflected light wave with the light sensor; and
comparing a parameter of the reflected light wave to a parameter of the at least one light wave to determine the oxygen saturation in the blood of the fetus.

2. The sensor system of claim 1, further comprising an oxygenator in fluid communication with the fetus so as to receive blood from the fetus,
wherein the sensor system determines the oxygen saturation of the blood of the fetus within the oxygenator.

3. The sensor system of claim 2, wherein the oxygenator introduces oxygen into the blood of the fetus, and
wherein control unit modifies an amount of oxygen supplied to the oxygenator to modify an amount of oxygen introduced into the blood of the fetus by the oxygenator.

4. The sensor system of claim 1, wherein the light source emits a first light wave at a first wavelength and emits a second light wave at a second wavelength different from the first wavelength.

5. The sensor system of claim 1, wherein the at least one light wave is at a wavelength ranging from about 400 nanometers to about 700 nanometers.

6. The sensor system of claim 5, wherein the light sensor senses a first reflected light wavelength and a second reflected light wavelength different from the first wavelength.

7. The sensor system of claim 6, wherein the light sensor senses the first and second reflected light wavelengths in response to the light source emitting a single light wave at a selected wavelength.

8. The sensor system of claim 6, wherein the light sensor senses the first and second reflected light wavelengths simultaneously.

9. The sensor system of claim 1, wherein the at least one memory has instructions stored therein that, upon execution by the control unit, causes the sensor system to perform operations comprising:
comparing a parameter of the reflected light wave to one or more stored values to determine the oxygen saturation in the blood.

10. The sensor system of claim 1, wherein the sensor system senses oxygen saturation levels ranging from about 30% to about 100%.

11. The sensor system of claim 10, wherein the sensor system senses oxygen saturation levels ranging from about 40% to about 75%.

12. A method for measuring oxygen saturation in a blood of the fetus in an ex-utero environment, the method comprising the steps of:
connecting a premature fetus to an ex-utero system configured to provide oxygen to the fetus, wherein the connecting step comprises the steps of attaching a first cannula to a vein of an umbilical cord, attaching a second cannula to an artery of the umbilical cord and connecting one or more of the first and second cannulae to an oxygenator such that blood is delivered from the fetus to the oxygenator and blood is delivered from the oxygenator to the fetus;
emitting, by a light source, a light wave toward the blood of the fetus;
sensing, by a light sensor, a reflected light wave reflected by the blood of the fetus; and
comparing, by a control unit, a parameter of the reflected light wave to one or more stored values to determine the oxygen saturation in the blood of the fetus.

13. The method of claim 12, wherein emitting the light wave includes emitting a light wave at a wavelength of about 400 nanometers to about 700 nanometers.

14. The method of claim 12, wherein the method includes determining the oxygen saturation in the blood of the fetus within the oxygenator.

15. The method of claim 12, wherein the method includes determining the oxygen saturation in the blood of the fetus without infrared light.

16. The method of claim 15, wherein the light source and the light sensor are each positioned on a same side of the oxygenator.

17. The method of claim 12, wherein the method includes:
modifying, by the control unit, an amount of oxygen supplied to the oxygenator thereby modifying the amount of oxygen introduced into the blood of the fetus by the oxygenator.

18. The method of claim 12, wherein the sensing step includes sensing, by the light sensor, a first reflected light wavelength and a second reflected light wavelength different from the first wavelength.

19. The method of claim 12, wherein the reflected light wave sensed in the sensing step is a first reflected light wave at a first wavelength and the method includes sensing, by the light sensor, a second reflected light wave at a second wavelength, and
wherein the first and second light waves are a reflection of the light wave emitted by the light source.
